SEQUENCE LISTING (2 sequences)

SEQ ID NO: 1 - RNA, 19 nt, synthetic construct
  misc_feature 1..19: note = luciferase siRNA
  source 1..19: mol_type = other RNA
cttacgctgagtacttcga

SEQ ID NO: 2 - RNA, 20 nt, synthetic construct
  misc_feature 1..20: note = hsa-miR-124a
  source 1..20: mol_type = other RNA
taaggcacgcggtgaatgcc

(19) United States
(12) Patent Application Publication
(10) Pub. No.: US 2025/0084414 A1
(43) Pub. Date: Mar. 13, 2025

(54) NONVIRAL VECTORS FOR GENE THERAPY

(71) Applicants: POLITECNICO DI MILANO, Milano (IT); FONDAZIONE IRCCS ISTITUTO NEUROLOGICO CARLO BESTA, Milano (IT)

(72) Inventors: Francesca BALDELLI, Milano (IT); Pierangelo METRANGOLO, Milano (IT); Gabriella CAVALLO, Milano (IT); Nina BONO, Milano (IT); Gabriele CANDIANI, Milano (IT); Marta ROSATI, Milano (IT); Giuseppe PINTER LAURIA, Milano (IT); Stefania MARCUZZO, Milano (IT)

(21) Appl. No.: 18/845,379

(22) PCT Filed: Mar. 6, 2023
(86) PCT No.: PCT/IB2023/052071

§ 371 Date: Sep. 9, 2024

(30) Foreign Application Priority Data
Mar. 9, 2022 (IT) 102022000004496

(51) Int. Cl. C12N 15/113; C08G 83/00

(52) U.S. Cl. CPC C12N 15/113; C08G 83/002; C12N 2310/141; C12N 2310/351; C12N 2320/32

(57) ABSTRACT

The present invention relates to fluorinated amphiphilic dendrimer structures (FJDs) capable of self-assembling into supramolecular systems of different size and shape. A further object is a supramolecular complex comprising at least one of the dendrimer structures according to the present invention and the use thereof in gene therapy.

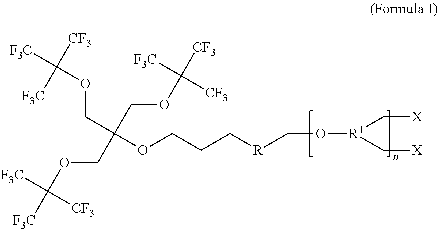

### BACKGROUND ART

Gene therapy is currently limited in clinical application also due to the difficulty of delivering genetic material into cells safely and efficiently.

The approaches used are based on viral and non-viral vectors. Viral vectors are very efficient but generally induce an immune response and require very complex preparation techniques. Non-viral vectors, generally consisting of lipids or cationic/ionizable polymers, are less efficient with respect to viral vectors, but less immunogenic and easier and more versatile to prepare. The presence of cationic polar heads on non-viral vectors also leads to some cytotoxicity.

Alongside an efficient and safe delivery system, another strongly felt need is to be able to non-invasively monitor the biodistribution of the vector, so as to verify its effective reaching of the target site.

Lipid nanoparticles for use as non-viral vectors are summarized by Kulkarni J A et al. in Nucleic Acid Therapeutics 2018; 28, 3.

Gaucheron J et al. in Bioconjugate Chem. 2001; 12, 6, 949-963 describe cationic lipid vectors functionalized with fluorinated glycerophosphoethanolamine.

Wang M et al, in Nat Commun 2014; 5, 3053 describe polymeric vectors with long perfluorinated linear chains.

Functionalization has been shown to favor the internalization of genetic material at the intracellular level and its release from endosomes to perform the desired biological function. However, to obtain the above results, linear perfluorinated chains have been used and a high density of conjugated fluorinated chains on a single polymer is required, resulting in a “congested” polymer surface and the impossibility of intervening with further modifications with other functional ligands. Furthermore, monitoring such vectors by clinical-level imaging techniques becomes difficult unless radiotracers involving complex and expensive preparations are used. In fact, the use of the ¹⁹F magnetic resonance technique (¹⁹F-MRI) is complicated by the fact that the perfluoroalkyl chains currently used produce multiple magnetic resonance signals due to the presence of magnetically non-equivalent fluorine atoms. This greatly compromises the sensitivity of the analysis. Lastly, the use of perfluoroalkyl chains presents considerable environmental sustainability problems, as compounds containing long fluorinated chains (more than six carbon atoms) have shown high persistence in the environment and high potential for bioaccumulation. Dendrimers are a class of highly branched macromolecular synthetic compounds which have repetitive structures.

The elements characterizing dendrimers are to be found in the architecture thereof, which comprises:
a central nucleus, defining the internal dimension, the number of branches and the direction;
layers of repetitive units (called generations) starting from the central nucleus and regulating the flexibility and size of the molecule;
active terminal groups, and thus with surface charge, which define the chemical properties thereof and the interaction possibility.

Dendrimers have been suggested as polymeric non-viral vectors (Dufès C et al. Dendrimers in gene delivery. Advance drug delivery reviews 2015; 57:2177-2202). For example, cationic dendrimers belonging to the class of polyamidoamines (PAMAM), polypropylene imines (PPI), poly-L-lysine carbosilanes (CBS) (PLL9) and phosphorus-containing dendrimers have been suggested for the delivery of siRNAs and microRNAs. Each of these classes has advantages and limitations, therefore the need to have non-viral vectors capable of overcoming the limits found with the ligands available to date remains strongly felt.

### DETAILED DESCRIPTION OF THE INVENTION

### Description of the Drawings

FIG. 1: A) ¹H-NMR spectrum of FDG₂N and peak assignment, B) ¹⁹F-NMR spectrum of FDG₂N. As shown by the integration, each cationic amphiphile molecule carries 27 fluorine atoms and 4 TFA anions as counterions. Solvent: CD₃OD.

FIG. 2: Cryo-EM images. A, B) 2.5 mM FDG₂N in 10 mM HEPES buffer (pH 7.4), fresh sample, presence of small micelles about 5-10 nm in diameter. C, D) 2.5 mM FDG₂N in 150 mM NaCl, fresh sample, coexistence of small micelles (10-20 nm in diameter) and larger spherical aggregates (about 50-70 nm).

FIG. 3: Cryo-EM images. A) 0.56 mM FDG₂N in 150 mM NaCl alone, fresh sample. The image shows the coexistence of aggregates of different sizes (10-70 nm in diameter). B) Dendriplexes obtained by dissolving FDG₂N in 150 mM NaCl at the concentration of 0.56 mM with N/P=30.

FIG. 4: Impact of dendriplex mimetic miR124a on survival of epSPCs. A) Optical microscope images of epSPCs treated with: negative control (NC) N/P5; NC N/P10; NC N/P20; NC N/P30; NC N/P40 or with miR-124a N/P5 dendriplex; miR-124a N/P10 dendriplex; miR-124a N/P20 dendriplex, miR-124a N/P30 dendriplex; miR-124a N/P40 dendriplex. Optical microscopic images of epSPCs B) under baseline conditions, C) treated with empty N/P40; D) treated with Lipofectamine and NC (above) or with Lipofectamine and miR-124a (below). The black arrows indicate areas in the plate which are lacking cells, likely due to cell death. Bar Scale: 50 μm. E) confocal microscopic images of epSPC cells in baseline conditions (left), treated with miR-124a N/P30 dendriplex (center), and with lipofectamine and miR-124a (right), labeled for the neural stem cell marker nestin (gray), and stained with DAPI (white) to highlight the nuclei. Bar scale=50 μm. The graph shows the quantification of nestin-positive epSPC cells under baseline conditions, treated with miR-124a N/P30 dendiplex and treated with lipofectamine and miR-124a; The data are expressed as mean number of nestin-positive cells±SD obtained by analyzing 6 fields per slide chosen at random. F) Real Time RT-PCR measurements of CASP6 gene expression levels in epSPCs under baseline conditions, treated with miR-124a N/P30 dendriplex and treated with lipofectamine and miR-124a (N=6 cultures per group). CASP6 expression levels are presented as mean±SE of relative values (2-ΔCt) normalized with the 18S housekeeping gene. Mann Whitney test*p<0.05.

FIG. 5: Modulation of miR-124a-mediated DLX2 gene expression. A) Real Time RT-PCR analysis to evaluate miR-124a expression in epSPC cultures under baseline conditions and after treatment with: NC N/P5-N/P40; lipofectamine (Lipo) and NC; miR-124a N/P5-N/P40 dendriplex; lipofectamine (Lipo) and miR-124a. The MiR-124a levels are presented as mean±SEM of relative values (2-ΔCt) normalized towards the endogenous U6 control. Mann Whitney test *p<0.05, **p<0.01 miR-124a N/P5-N/P40 dendriplex and lipofectamine (Lipo) and miR-124a with respect to the baseline condition; ###p<0.001, miR-124a N/P5-N/P40 dendriplex and Lipo and miR-124a with respect to the negative control N/P5-N/P40 and Lipo and NC. B) Real Time RT-PCR analysis to evaluate DLX2 expression in epSPC cultures under baseline conditions and after treatment with: NC N/P5-N/P40; lipofectamine (Lipo) and NC; miR-124a N/P5-N/P40 dendriplex; lipofectamine (Lipo) and miR-124a. The DLX2 mRNA levels are presented as mean±SEM of relative values (2-ΔCt) normalized towards the 18S housekeeping gene. Mann Whitney test **p<0.01, miR-124a N/P30 dendriplex with respect to the baseline conditions: ##p<0.01, miR-124a N/P30 dendriplex towards NC N/P30; $$$ p<0.001, miR-124a N/P30 dendriplex to Lipo and miR-124a.

For the purposes of the present description, “dendriplex” means a carrier comprising at least one dendrimer structure and at least one nucleic acid.

“Lipoplex” means a structure comprising lipofectamine and at least one nucleic acid.

In a dendriplex, “N/P” denotes the ratio of the nitrogen atoms of the dendrimer structure to the phosphorus atoms of the nucleic acid charged therein, in a preferred form of the miRNA.

The present invention first relates to fluorinated amphiphilic dendrimer structures (FJDs) capable of self-assembling into supramolecular systems of different size and shape. The structures of the invention have the general formula (I) and comprise a fluorinated hydrophobic portion and a polyester-based hydrophilic portion.

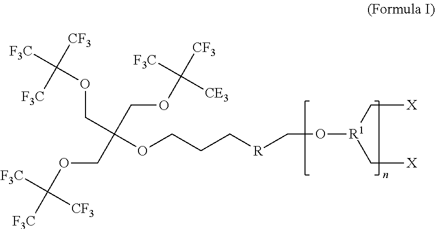

In Formula (I)
n is an integer between 1 and 5;
R is selected from

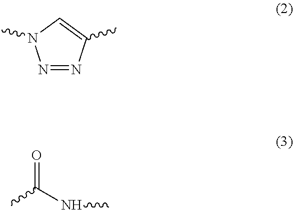

R¹ is selected from

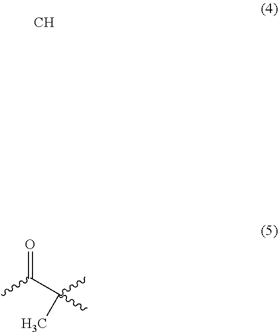

X is selected independently from:
   OH; (6)
   COOH (7)
   NH₂ (8)
   NH₃⁺Y⁻ (9)
   NR²₂ (10)
   NR²₃_{⁺}, Y⁻ (11)
where
R² is selected independently from —CH₃, —CH₂CH₃, —CH₂CH₂CH₃, —CH(CH₃)₂, —CH₂OH, —CH₂CH₂OH;
Y⁻ is selected from:

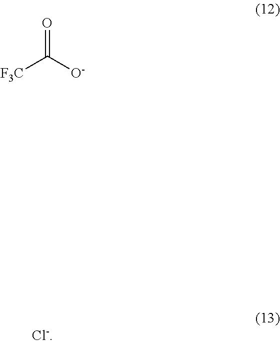

In an embodiment, n is 2 or 3, preferably it is 3.

In an embodiment, R is

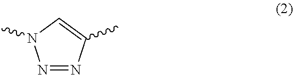

In an embodiment, R¹ is

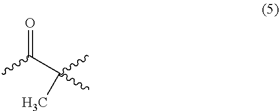

In an embodiment, X=NH₃⁺Y⁻ and Y is selected from

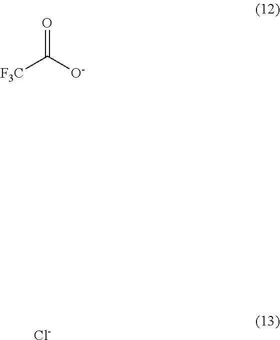

preferably Y is

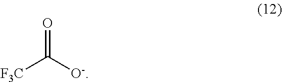

In an embodiment, n=3,

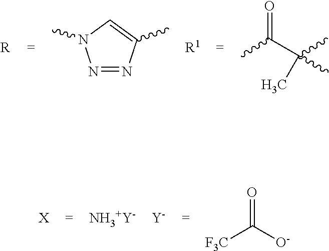

In this embodiment, said compound is referred to as FDG₂N.

In an embodiment, n=3,

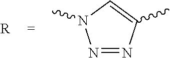

### R¹=CH; X=OH

The present invention further relates to supramolecular complexes comprising at least one of said dendrimer structures and one or more nucleic acids.

The present invention further relates to a composition comprising at least one of said dendrimer structures, an effective amount of a nucleic acid and a pharmaceutically acceptable vector.

Said nucleic acids are selected from both single and double-stranded deoxyribonucleic acid (DNA), ribonucleic acid (RNA), ribosomal RNA (rRNA), catalytic RNA (cRNA), snRNA, messenger RNA (mRNA), transfer RNA (tRNA), siRNA, shRNA, protein nucleic acids (PNA) and substituted nucleic acid oligonucleotides.

In a preferred form, said nucleic acid is a nucleic acid capable of mediating the RNA interference (RNAi) in which the nucleic acid is an RNA molecule selected from the group consisting of an siRNA and an shRNA.

In a preferred form, said nucleic acid is a mimetic miR-124a, i.e., a chemically modified double-stranded RNA molecule designed to mimic endogenous microRNA.

In a preferred form, said dendrimer structure is FDG₂N.

In an embodiment, a pharmaceutical formulation comprising the composition described herein is claimed.

The present invention further relates to a method for obtaining said supramolecular complex, where said method comprises providing a dendrimer structure of Formula (I) and dispersing it in a saline aqueous solution with nucleic acids. Said dendrimer structure of Formula (I) and said nucleic acids are dispersed in molar ratio between 50 and 600, in a preferred embodiment 344, i.e., expressing said ratio as NIP, it is between 5 and 40, in an embodiment it is 30.

The present invention further relates to one or more of the supramolecular complexes described for use in gene therapy.

In an embodiment said use is in the treatment of neurological/neurodegenerative diseases.

In an embodiment, said supramolecular complex comprises miR-128 and miR-15 and said complex is for use in the treatment of Alzheimer's disease.

In an embodiment, said supramolecular complex comprises miR-30 and miR-26a and said complex is for use in the treatment of Parkinson's disease (Chakraborty et at, J. Adv. Res. 2021; 28: 127-138).

In an embodiment, said supramolecular complex comprises miR-206 and miR-146a and said complex is for use in the treatment of amyotrophic lateral sclerosis (Rinchetti et al., Mol. Neurobiol. 2018; 2617-2630).

In an embodiment, said supramolecular complex comprises miR-19a and miR-19b and said complex is for use in the treatment of multiple sclerosis (Gao et al., Clin. Chim. Acta. 2021; 92-99).

The present invention further relates to a supramolecular complex according to the present invention for use in tracking dendriplex after the administration thereof.

MiR-124a regulates and induces neuronal differentiation in the adult brain and spinal cord by positively targeting the Distal-Less Homeobox 2 gene (DLX2) (Marcuzzo et al, Exp Neurol. 2014; 91-101; Marcuzzo et al., Mol. Brain 2015; 8, 5). In ependymal stem/progenitor cells (epSPCs) (Haidet-Phillips et al., Nat. Biotechnol. 2011; 824-828; Marcuzzo et at, 2014 cit.) residing in the adult spinal cord, miR-124a is involved in the signaling pathways underlying neurogenesis processes in the spinal cord. The present inventors have thus surprisingly observed that a mimetic miR-124a, administered by the supramolecular complex according to the present invention, is capable of increasing the expression levels of miR-124a in ependymal stem/progenitor cells, without prematurely activating apoptosis, as is instead observed when the same is administered by lipofectamine. The results obtained indicate that the use of the supramolecular complex according to the present invention is an effective method for obtaining mi-RNA-mediated gene regulation.

### Advantages

The dendrimer structures according to the present invention allow to make a multiplicity of equivalent fluorine atoms (27 F) available, useful for example for ¹⁹F-MRI purposes, together with a stable and dense packaging, due to branched fluorinated chains' intrinsic tendency to crystallize.

Furthermore, the presence of four ethereal bonds in the nucleus accelerates the degradation of the molecule in the environment, thus overcoming the bioaccumulation problems of PFAs.

Such molecules have shown a finely controllable assembly in aqueous medium, as a function of the equilibrium generated between the two domains, fluorinated and hydrophilic.

The supramolecular complexes according to the present invention have surprisingly shown a higher transfection capacity with respect to that observed using lipid non-viral vectors, associated with significantly reduced cytotoxicity. The in vitro and in vivo results confirm the validity of the approach for the delivery of nucleic acids for the purpose of gene therapy, even where the target is in cells of the nervous system.

The presence of ¹⁹F in the complexes according to the present invention advantageously allows the location thereof to be traced when administered in an organism.

### EXAMPLES

### Example 1: Synthesis of First, Second and Third Generation Fluorinated Amphiphilic Dendrimers

For the synthesis, the chemicals used as reagents and solvents were used as received without further purification and purchased with purity>97% from: TCl Deutschland GmbH: Sigma Aldrich, DE; Fluorochem, U.K.

TLC thin layer chromatography was conducted on plates pre-coated with Si 60-F254 silica gel (Merck, Darmstadt, Germany). Flash chromatography was performed on J.T. Baker silica gel mesh size 230-400 and solutions of ninhydrin or potassium permanganate were used as chemical dyes.

The synthesis was carried out following a convergent procedure requiring the separate synthesis of the fluorinated derivative and the hydrophilic part, based on small generation polyester dendrons (1st, 2nd and 3rd) with 2,2-Bis(hydroxymethyl)propionic acid (BIS-MPA) as monomer.

The synthesis of the branched fluorinated structure was optimized and carried out so as to obtain the azide derivative suitable for bonding with the polyester part.

As shown in diagram 1, the synthesis of the azide derivative (F27-N3) starts from pentaerythritol (1 equivalent, 100 g) which is reacted with tert-butyl acrylate (1.2 equivalents) in the presence of NaOH (0.2 equivalents) as base in dimethyl sulfoxide (DMSO, total volume: 128 ml) as solvent. The compound a (1 equivalent, 1.36 g) is then reacted through Mitsunobu reaction with perfluoro-tert-butyl alcohol (6 equivalents) in the presence of triphenylphosphine (PPh₃) and diisopropyl azodicarboxylate (DIAD) (6 equivalents each) in dry tetrahydrofuran (THF, total volume: 38 ml). The fluorinated ester, compound b (1 equivalent, 0.4 g), is then reduced to obtain the alcohol derivative in the presence of LiAlH₄ (4 equivalents) in anhydrous THE (total volume: 50 ml).

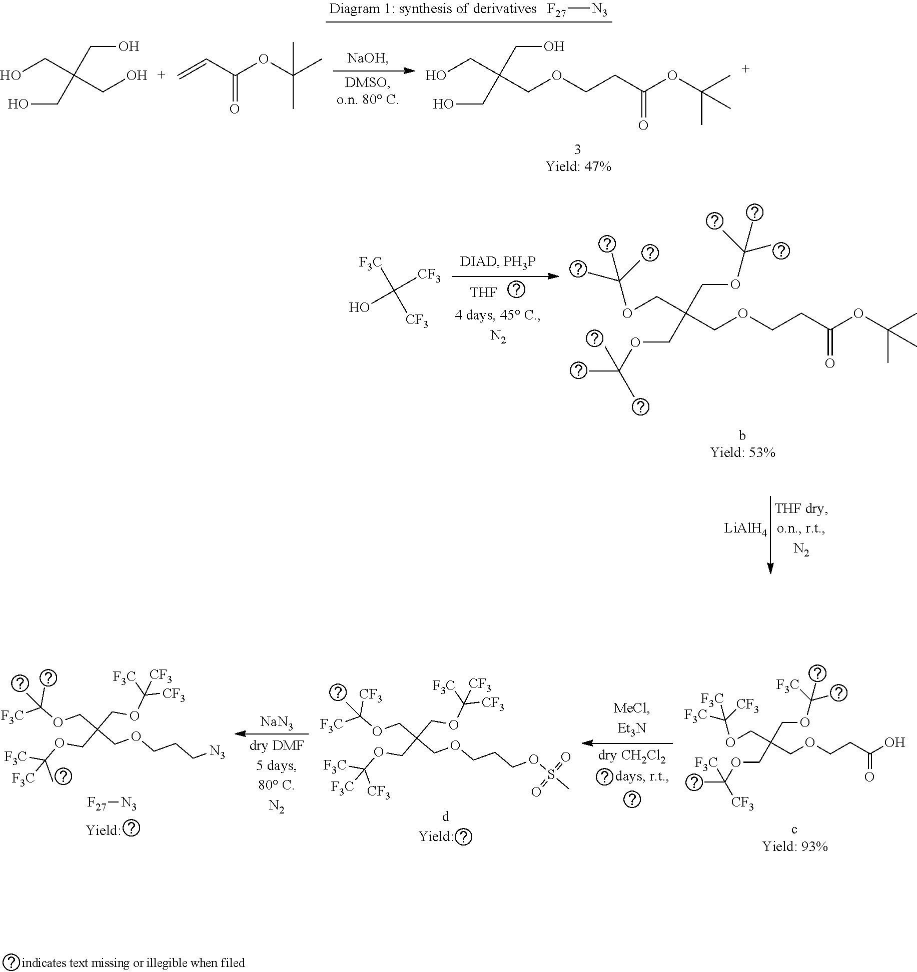

Compound c (1 equivalent, 0.85 g) is converted to the mesylated derivative through a substitution reaction in the presence of methanesulfonyl chloride (MsCl, 3 equivalents) and triethylamine (Et₃N, 3 equivalents) as base in anhydrous dichloromethane (CH₂Cl₂, total volume: 26 ml). Finally, the derivative d (1 equivalent, 2.90 g) can be converted to F₂₇—N₃ by a second substitution reaction in the presence of sodium azide (2.2 equivalents) in anhydrous dimethylformamide (DMF, total volume: 19 ml). The compound a was purified by flash chromatography on silica gel using diethyl ether and acetone (1:1) as eluent (Rf=0.3) and permanganate solution as TLC chemical dye. All other intermediates could be purified by extraction from water with organic solvents (mainly dichloromethane or hexane) without further purification. Compound b was recrystallized from ethanol to remove all by-products.

The synthesis of polyester dendrimers was carried out separately starting from the protection of the OH groups of Bis-MPA, as reported in diagram 2.

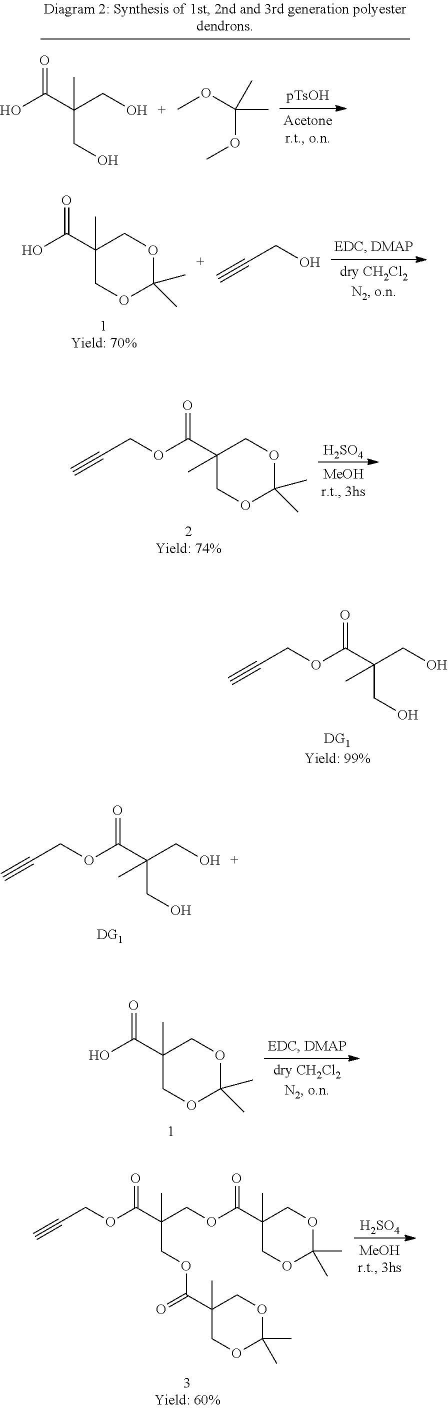

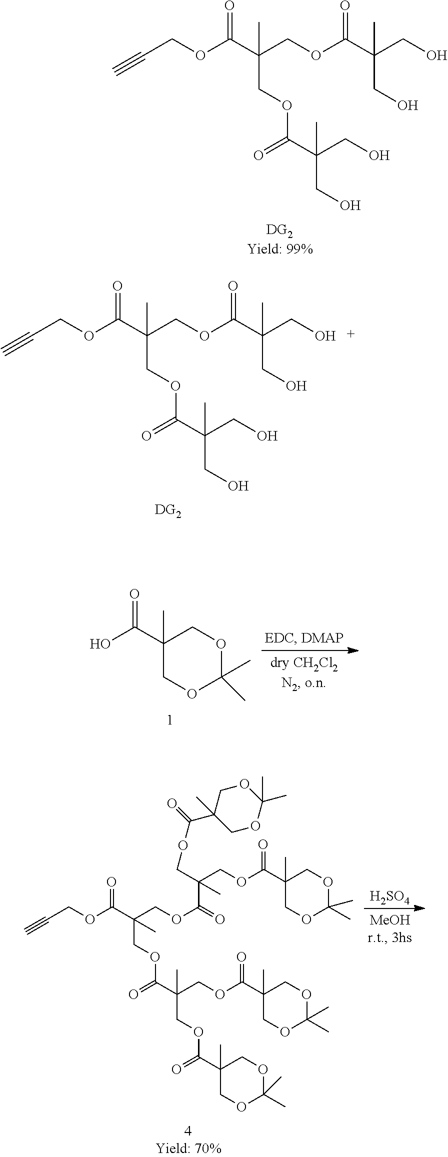

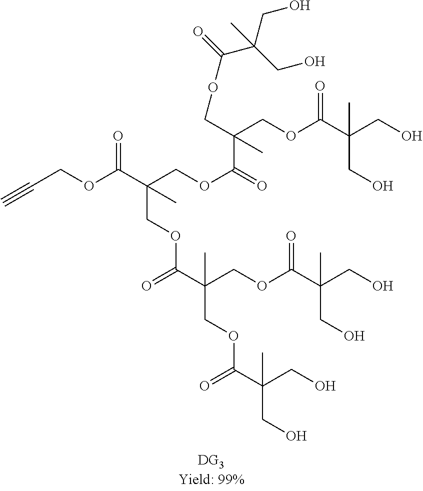 [SMILES: C#CCOC(=O)C(C)(COC(=O)C(C)(COC(=O)C(C)(CO)CO)COC(=O)C(C)(CO)CO)COC(=O)C(C)(COC(=O)C(C)(CO)CO)COC(=O)C(C)(CO)CO]

The reaction is conducted in the presence of Bis-MPA (1 equivalent, 5 g), 2,2-dimethoxypropane (2 equivalents) and a catalytic amount of para-toluenesulfonic acid (pTsOH, 0.1 equivalents) in acetone (total volume: 25 ml). Then the propargyl ester is obtained by exploiting Steglich esterification, Therefore, the compound 1 (1 equivalent, 252 mg) is reacted with propargyl alcohol (2 equivalents) in the presence of 1-ethyl-3-(3-dimethylaminopropyl) carbodiimide hydrochloride (EDO, 1.1 equivalents) and 4-dimethylaminopyridine (DMAP, 0.1 equivalents) in anhydrous CH₂Cl₂ (total volume: 10 ml). Acetonide deprotection is carried out in the presence of compound 2 (1 equivalent, 151 mg) and sulfuric acid (H₂SO₄, 0.85 equivalents) in methanol (total volume: 3 ml) is to obtain the 1st generation polyester dendron (DG1), Generation growth is obtained by iterative alternation of Steglich esterification with acetonide deprotection. Therefore, in order to obtain the 2nd generation polyester dendrone (DG2), DG1 (1 equivalent, 112 mg) is reacted with the compound 1 (5 equivalents) in the presence of EDO and DMAP (5 and 0.5 equivalents, respectively) in anhydrous CH₂Cl₂ (total volume: 10 ml); then the compound 3 (1 equivalent, 170 mg) is deprotected in the presence of H₂SO₄ (1.7 equivalents) in methanol (total volume: 5 mL). Similarly, DG2 (1 equivalent, 150 mg) is reacted with the compound 1 (8 equivalents) in the presence of EDC and DMAP (8 and 0.5 equivalents, respectively) in anhydrous CH₂Cl₂ (total volume: 13 mL) to achieve intermediate 4. Compound 4 (1 equivalent, 200 mg) is then deprotected acetonide in the presence of sulfuric acid (3.4 equivalents) in methanol (total volume: 7 ml) to obtain the 3rd generation dendron (DG3). Intermediates 2, 3 and 4 were purified by silica flash column chromatography using hexane and ethyl acetate (8:2) as eluent, in the case of 2, or hexane and ethyl acetate (7:3) for 3 and 4. In order to connect the branched fluorinated part and the hydrophilic polyester part through a rigid linker, the copper-catalyzed azide-alkyne cycloaddition (CuAAC) reaction was carried out as shown in diagram 3.

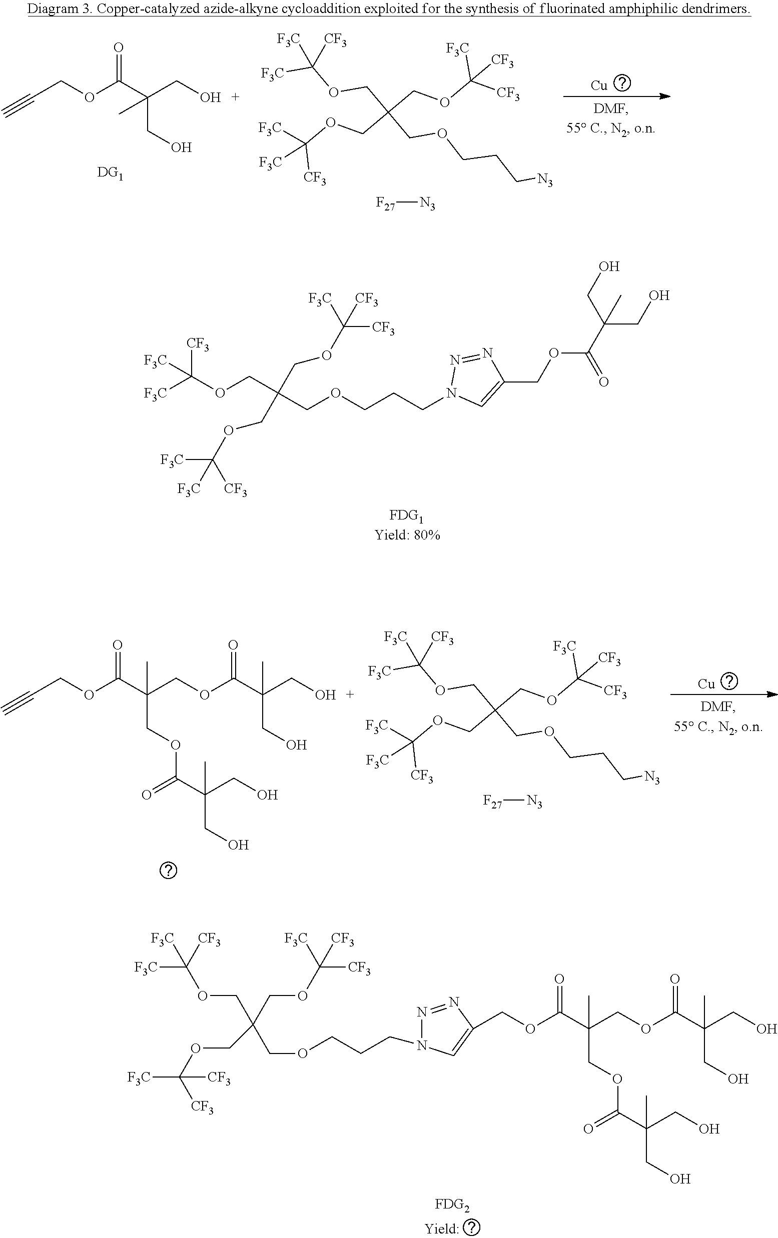

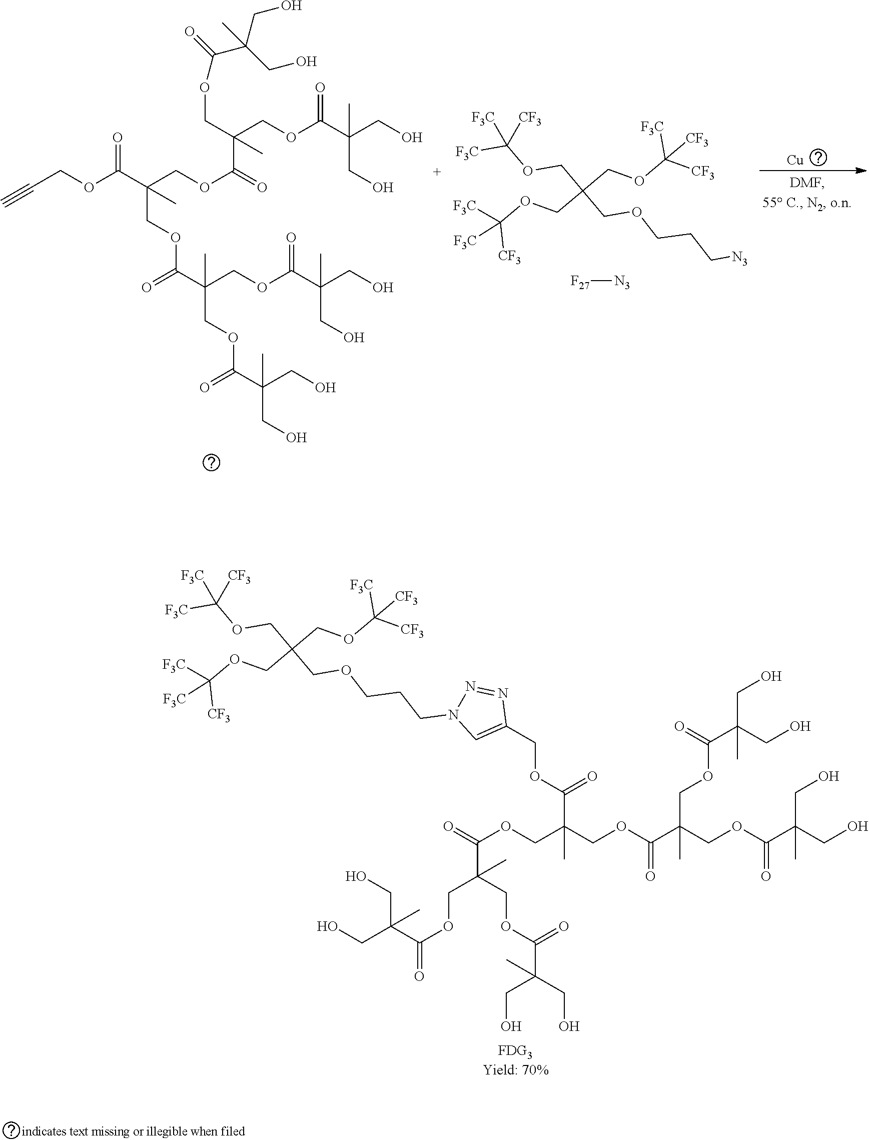

This strategy allowed the orthogonal connection of the two portions with good yields and without side products. The synthesis of the first-generation fluorinated amphiphilic dendrimers (FDG₁) was carried out by dissolving compound DG₁ (1 equivalent, 313 mg) and copper (I) acetate (0.1 equivalents) in DMF. F₂₇—N₃ (1 equivalent) was then dissolved in another DMF (total volume: 3 mL) and added to the reaction mixture. Similarly, the synthesis of the second-generation fluorinated dendrimer (FDG₂) was achieved by dissolving DG₂ (1 equivalent, 110 mg) and copper (I) acetate (0.15 equivalents) in DMF. F₂₇—N₃ (1 equivalent) was then dissolved in DMF (total volume: 3 mL) and added to the reaction mixture. Finally, FDG₃, the 3rd-generation fluorinated dendrimers, was obtained by mixing DG₃ (1 equivalent, 53 mg) and copper(I) acetate (0.5 equivalents) in DMF (total volume: 2.5 ml) and then adding F₂₇—N₃ (1 equivalent). All the CuAAC reactions were carried out at 55° C. under an inert atmosphere overnight. The reaction was then stopped, added to ice water and extracted with CH₂Cl₂; the organic phase was then washed twice with a 0.1% disodium EDTA solution in deionized water to remove copper and once with a saturated NaCl solution. The organic phase was collected, dried with Na₂SO₄ and rotary evaporated to obtain the compounds of interest. ¹H, ¹³C and ¹⁹F-NMR combined with ATR-FTIR and HRESI-MS analyses confirmed the formation of the final dendritic structures.

### Example 2: Synthesis of Second-Generation Fluorinated Amphiphilic Dendrimers for Gene Release Applications

To construct a gene release vector capable of binding the positive charges of the phosphate groups present in nucleic acids, the surface groups present in FDG₂ were chemically modified by inserting four primary ammonium salts. The synthetic steps are highlighted in diagram 4.

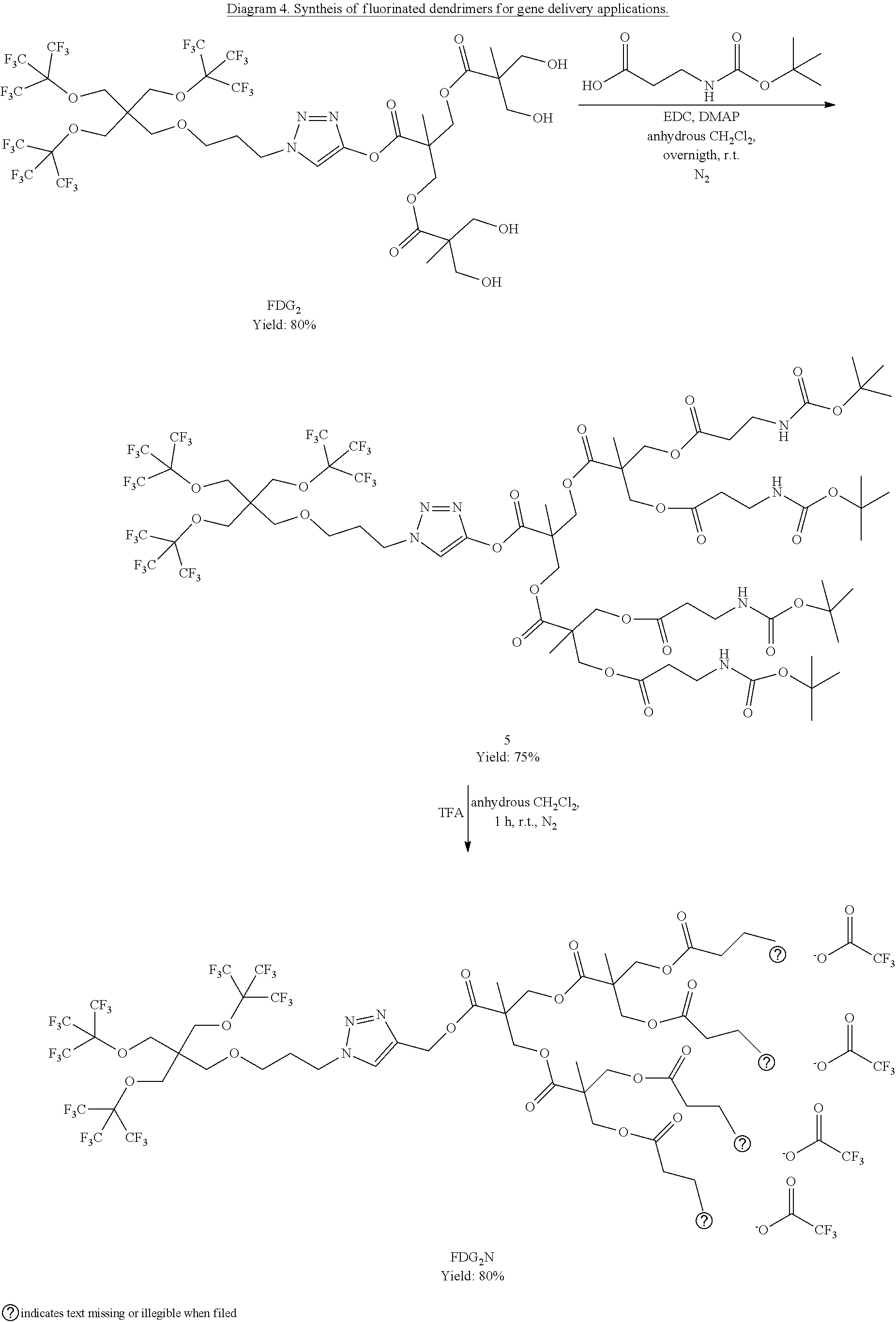

FDG₂ (1 equivalent, 176 mg) was reacted with Boc-beta-alanine (12 equivalents), EDC (12 equivalents) and DMAP (0.5 equivalents) in anhydrous CH₂Cl₂ (total volume: 7 ml) under an inert atmosphere overnight to obtain intermediate 5. To purify the product, flash silica column chromatography was carried out using a mixture of hexane and ethyl acetate (1:1) as eluent and a ninhydrin solution as TLC chemical dye (rf: 0.3). To remove the excess Boc-beta-alanine, the purified mixture was dissolved in CH₂Cl₂ and washed twice with 10% aqueous NaHCO₃ solution. The organic phase was dried over Na₂SO₄ and a pale-yellow oil was recovered after removal of the solvent by rotary evaporation. To achieve the tetra ammonium salt, compound 5 (1 equivalent, 195 mg) was dissolved in CH₂Cl₂ (total volume: 2 mL) and reacted with trifluoroacetic acid (TFA, 2 mL).

The reaction was carried out until total conversion of the precursor, confirmed by TLC (eluent: a mixture of hexane and ethyl acetate 1:1). After rotary evaporation of the reaction solvents, the compound was then solubilized in hexafluoro-2-propanol and dried again three times to remove the excess TFA. Finally, the product was dissolved in water and lyophilized to obtain FDG₂N. ¹H, ³C and ¹⁹F-NMR combined with ATR-FTIR and HRESI-MS analyses confirmed the formation of the final dendritic structure.

### Example 3: Characterization of Second-Generation Fluorinated Amphiphilic Dendrimers in Solution

FDG₂N solutions were obtained by directly dispersing the amount of solid necessary to obtain the desired concentrations (2.5 mM and 0.5 mM) in the final solvent: MilliQ water, 150 mM NaCl and 10 mM HEPES (4-(2-hydroxyethyl)-1-piperazine ethanesulfonic acid) the pH of which was adjusted with a 1M NaOH solution until pH=7.4 was reached. The solutions were aged at constant temperature (25° C.) and analyzed after 1 hour from sample preparation, after 24 hours and 48 hours of aging as previously discussed. The dispersions were analyzed by dynamic light scattering (DLS), Z potential and ¹⁹F-NMR. The multi-angle DLS was measured with the ALV compact goniometer system, provided with ALV-5000/EPP correlator, with He—Ne laser (λ=633 nm, output power 22 mW) as light source. The temperature was controlled with a thermostatic bath and set at 25° C. A volume between 800 μL and 1 ml was used for the analysis. DLS was measured at different time points (0, 24, 48 h) and diffusion angles θ=70-130° in 20° increments. Each measurement was the result of the average of three successive tests of 10 seconds each, with a threshold sensitivity of 10%, The data analysis was carried out with ALV-Correlator software. The apparent hydrodynamic rays at different angles were obtained by an intensity-weighted and number-weighted adaptation of the autocorrelation function. The hydrodynamic rays (RH) and polydispersion indices (PdI) were calculated using cumulative coupling. For a more accurate analysis, excluding cumulative adaptation due to the high polydispersion of the samples, CONTIN analyses were carried out. The Z potential was measured at 25° C. in folded capillary cells (U-shaped cells with two gold-plated beryllium/copper electrodes at the top) 48 hours after preparation of the colloidal dispersion with a Zetasizer Nano ZS (Malvern Instrument, Malvern, Worcestershire, UK), provided with a 633 nm laser. Before each measurement, the cells were cleaned with MilliQ water and then filled with about 1 ml of sample solution. The ¹⁹F-NMR spectra were performed by analyzing 500 μL of 2.5 and 0.5 mM FDG₂N dispersions mixed with 50 μL of deuterated water. The spectra were collected by setting 256 scans as input parameter. The peak of the TFA anions was set at −76.55 ppm. The measurements of T1 and T2 were obtained on the 2.5 mM FDG₂N solution in MilliQ water. The data adaptation was carried out by a single exponential adaptation and the raw data was analyzed by Bruker TopSpin software and MestReNova software. The form of the aggregates was further confirmed by Cryo-TEM.

The determination of critical micellar concentration (CMC) was carried out in both water and 150 mM NaCl, determining the pyrene fluorescence. Basically, a small aliquot (17 μl) of a pyrene solution (6.25 μM in methanol) is transferred to FDG₂N solutions at increasing concentrations up to a final volume of 1 mL (final pyrene concentration: 100 nM). The fluorescence emission and excitation spectra were obtained with a commercial spectrofluorometer (Jasco, FP˜6500).

To better understand the effect on self-assembly of FDG₂N of the nucleic acid bond, Cryo-EM images of dendriplexes (complexes obtained between FDG₂N and nucleic acids) were obtained, Briefly, the FDG₂N solution (0.56 mM) in 150 mM NaCl was mixed with siRNA (double strand, sequence SEQ ID NO: 1 CUUACGCUGAGUACUUCGA, coding for luciferase) at different nitrogen-phosphorus (N/P) ratios, in particular 20, 30 and 40, to mimic the in vitro conditions discussed below. The features of the aggregates were also confirmed by DLS, potential Z and NMR experiments.

### Example 4: Synthesis of FDG₂N and Characterization in Solution

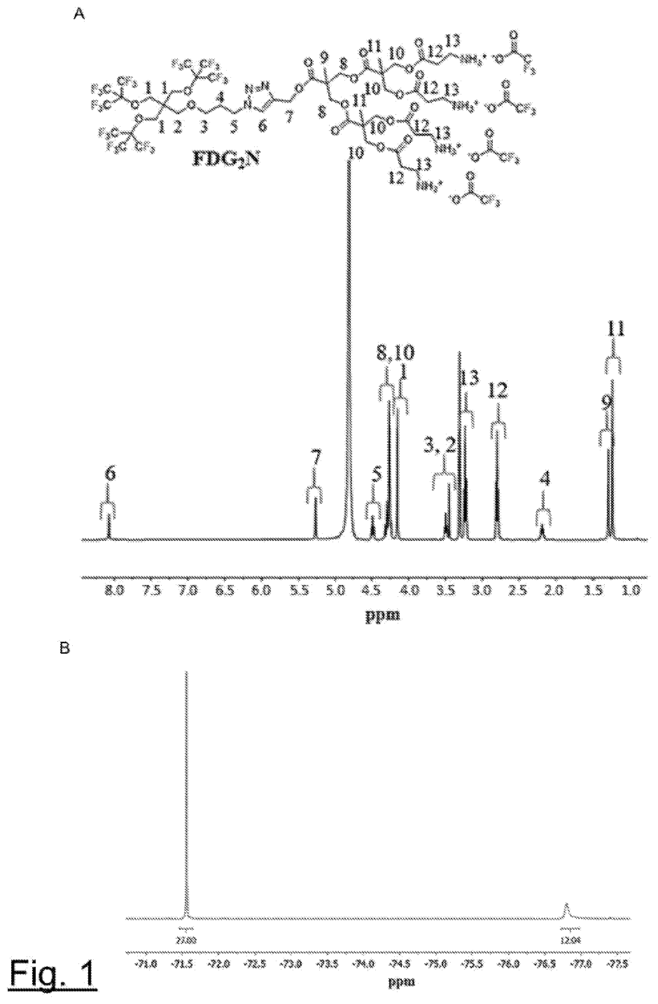

The synthetic procedure adopted allowed the isolation of the cationic fluorinated dendrimer FDG₂N with good yield and purity, as confirmed by the experiments ¹H and ¹⁹F-NMR and highlighted in FIGS. 1A and 1B, respectively.

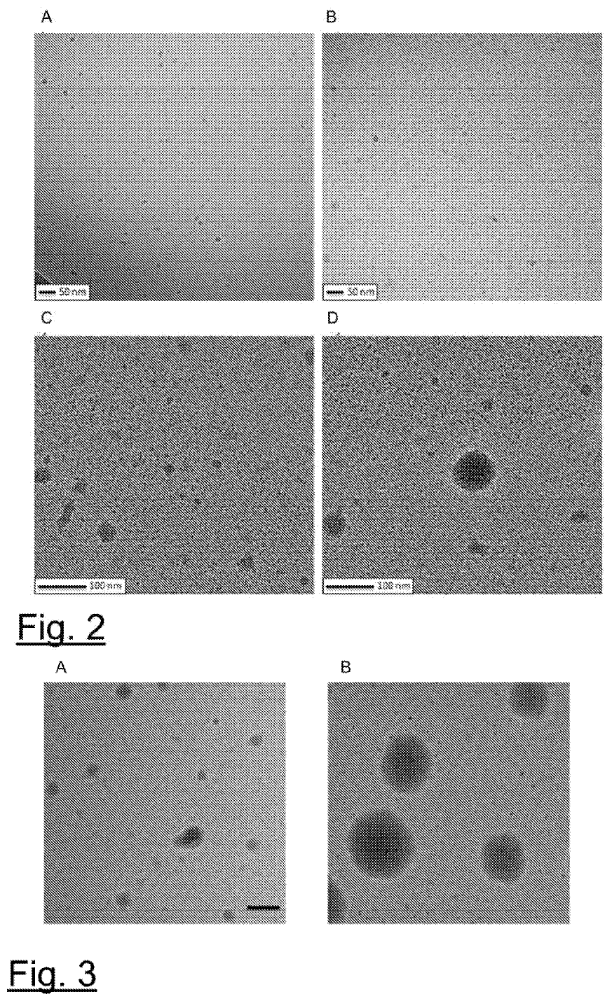

The compound is directly dispersible in aqueous media where it tends to self-assemble with CMC less than 50 μM in pure water and 20 μM in NaCl. In pure water and at physiological pH (10 mM HEPES Buffer, pH=7.4) it self-assembles mainly forming small micelles with an average hydrodynamic radius of about 2.5 nm, as confirmed by Cryo-TEM analyses and in accordance with the DLS results. When dissolved in 150 mM NaCl, FDG₂N tends to form larger spherical aggregates. In fact, near small micelles (15-20 nm in diameter), larger spherical aggregates (50-100 nm in diameter) can be observed. These larger aggregates are likely responsible for the higher hydrodynamic rays obtained in DLS in NaCl with respect to those observed in HEPES buffer and water. FIG. 2 shows the Cryo-TEM results observed in HEPES buffer (2A, 2B) and in 150 mM NaCl (2C, 2D).

The measurements of the Z potential reveal that even after 48 h the aggregates are positively charged with values above+40 mV suggesting the possibility of binding the nucleic acids.

The relaxation times T1 and T2 were determined in pure water for a concentration of 2.5 mM FDG₂N. Under these conditions, FDG₂N showed a T1 of 465 ms and a T2 of 85.4 ms, optimal for ¹⁹ F-MRI applications.

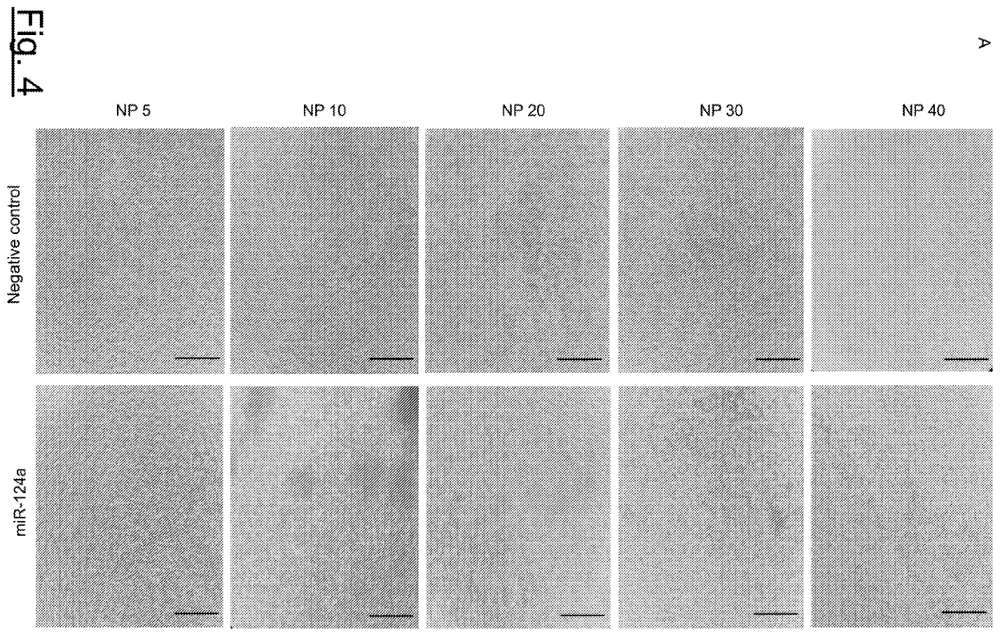

Complexation with siRNA was confirmed by Cryo-EM, where it was observed that the presence of siRNA influences the aggregation behavior of FDG₂N in solution, causing the formation of spherical aggregates of larger dimensions (FIG. 3B) with respect to those observed for the fluorinated dendrimer alone at the same concentration (FIG. 3A).

### Example 5: Preparation of Mimetic FDG₂ N-miR124a Dendriplex

2 μl of the miRNA of interest is diluted in 125 μl of Opti-MEM Medium for each well to be treated. To this 0.5 μM miRNA solution is added 125 μl of the reagent LipofectamineRNAiMAX Reagent (Cat. no. 13778-075 Thermo Fisher) as a control or 125 μl of FDG₂N at the concentrations indicated in Table 1. The miRNA used is hsa-miR-124-3p accession number MI0000443 (Mature miRNA Sequence SEQ ID NO: 2 UAAGGCACGCGGUGAAUGCC).

The dendriplexes are thus obtained at the N/P and molar ratios indicated in table 1, i.e., miR-124a N/P5 dendriplex, miR-124a N/P10 dendriplex, miR-124a N/P20 dendriplex, miR-124a N/P30 dendriplex, miR-124a N/P40 dendriplex used in the following examples.

TABLE 1

| N/P | initial [FDG₂N] (μM) | final [FDG₂N] (μM) |
|---|---|---|
| 5 | 28.72 | 14.36 |
| 10 | 57.4 | 28.7 |
| 20 | 114.4 | 57.2 |
| 30 | 172 | 86 |
| 40 | 229.6 | 114.8 |

### Example 6: Mimetic FDG₂N-miR124a Dendriplex does not Alter the Survival of epSPC

epSPCs (adult spinal cord-derived stem progenitor ependymal cells) were isolated from the spinal cord of 18-week-old mice.

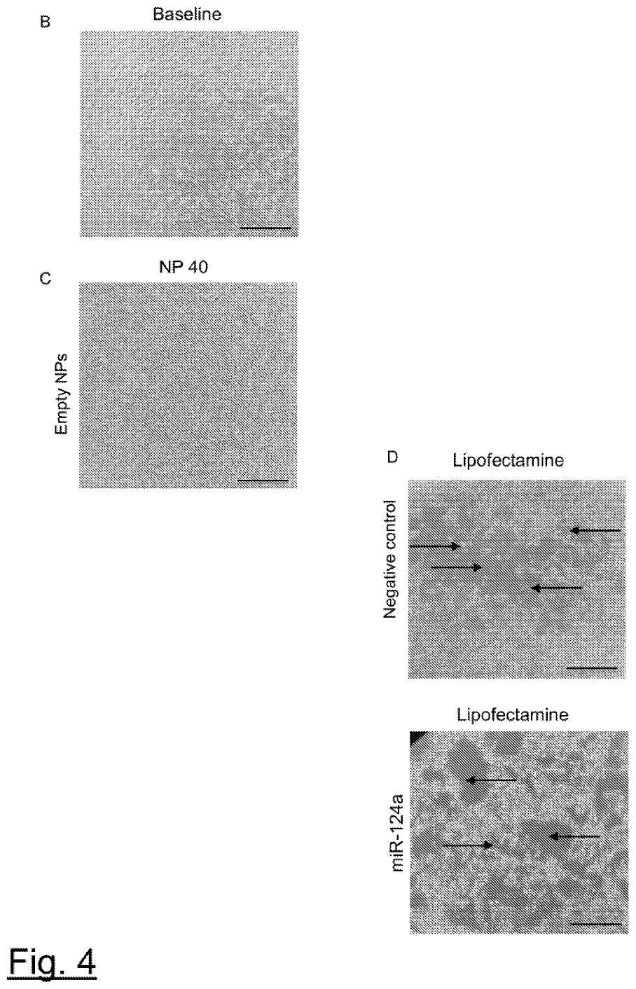

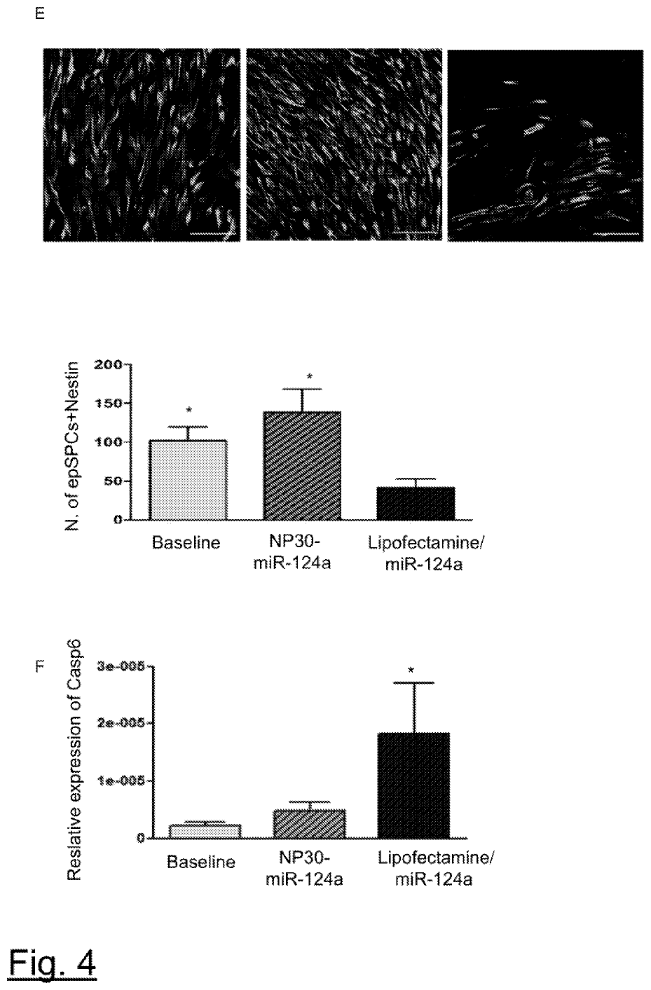

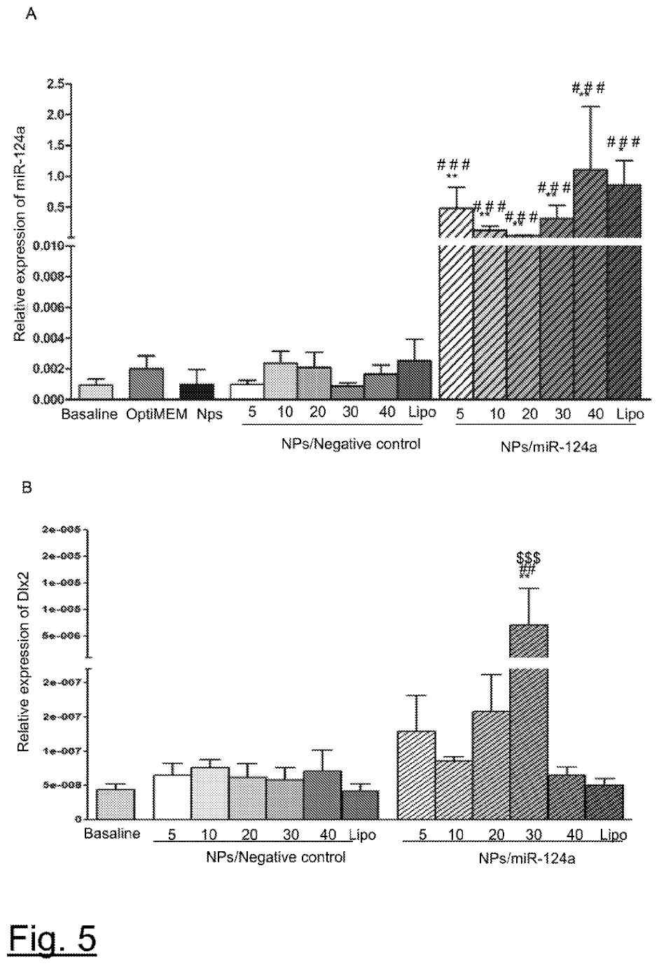

All the animal experiments were carried out in accordance with EU Directive 2010/63, and Italian law (law decree 26/2014) on the protection of animals used for scientific purposes. Control male mice B6.SJL were purchased from Charles River Laboratories, Inc. (Wilmington MA, USA), maintained and raised in compliance with institutional guidelines. The mice were sacrificed for tissue harvesting at 18 weeks of life by CO₂ exposure. After removal of the meninges and blood vessels, the spinal cord was cut into small pieces, dissociated with 0.05% collagenase I for 15 minutes at 37° C. and then processed to produce epSPC neurospheres, as described in Marcuzzo et al., 2014. On day 7, the epSPC neurospheres were dissociated into individual cells (cell passage 1, P1) and cultured for another week. This was repeated until day 21 (P3) of in vitro cultures, to obtain sufficient cells for further analyses. The neurospheres were monitored periodically by light microscopy (Eclipse TE 2000-S, Nikon, Tokyo, Japan). At P3, epSPCs were cultured at the density of 8×10⁴ in proliferative medium under different growth conditions: 1) baseline condition; 2) Opti-MEM condition corresponding to baseline condition but in the presence of Opti-MEM transfection medium; 3) negative control (NC) N/P5 consisting of a FDG₂N dendriplex charged with a molecule with random miRNA mimetic sequence (Thermo Fisher Scientific Inc., Foster City, MA, USA; in nitrogen-phosphorus ratio equal to 5; 4) NC N/P10; 5) NC N/P20; 6) NC N/P30; 7) NC; 8) Lipofectamine and NC; 9) MiR-124a N/P5 dendriplex consisting of FDG₂N charged with SEQ ID: 2 in nitrogen-phosphorus ratio equal to 5; 10) MiR124a N/P10 dendriplex; 11) MiR-124a N/P20 dendriplex; 12) MiR-124a N/P30 dendriplex; 13) MiR-124a N/P40 dendriplex; 13) Lipofectamine and miR-124a; 14) N/P40 empty. The cells were maintained in culture for 72 hours. epSPCs were then collected for molecular and immunofluorescence analyses. Exemplary images of what was observed with a fluorescence microscope under the conditions indicated are shown in FIG. 4A-D.

After the dendriplex treatments, the density of epSPCs was similar under all culture conditions. Conversely, the cells cultured in the presence of lipofectamine, negative control (NC) or mimetic miR-124a, showed a reduced cell density with respect to that observed in the absence of lipofectamine. To better assess the impact of the dendriplexes and lipofectamine on epSPC cultures, immunofluorescence staining was performed for nestin, a marker of neural stem/progenitor cells, under baseline conditions and after treatment with miR-124a N/P30 dendriplex or mimetic miR-124a lipofectamine.

For this purpose, epSPC neurospheres were dissociated into individual cells, plated on Matrigel-treated coverslips at the density of 8×10⁴ and maintained 72 hours in proliferative medium under the following conditions: 1) baseline; 2) miR-124a N/P30 dendriplex; and 3) miR-124a mimetic lipofectamine. They were then fixed in 4% paraformaldehyde at room temperature for 20 minutes, permeabilized with 0.1% Triton X-100 and treated with 10% anti-goat in PBS to block the non-specific binding sites. The samples were then incubated with anti-mouse nestin (Mouse-antimouse Nestin IgG, 1:200, Millipore, Billerica, MA). The immunopositivity was revealed with anti-mouse IgG conjugated with Alexa Fluor 488 (Thermo Fischer Scientific). The cells were stained with 4,6-diamidino-2-phenylindole (DAPI) and the coverslips were mounted with FluorSave. Confocal fluorescence images were obtained with a laser scanning microscope (Eclipse TE 2000-E, Nikon) and analyzed using EZ-C1 3.70 imaging software (Nikon). The quantitative evaluation of the individual nestin-positive cells was carried out on 6 randomly selected fields per X60 magnified slide for each condition using ImageJ software (version 1.52 p). Exemplary confocal images obtained under the three different conditions tested are shown in FIG. 4E. The quantification of the results obtained is shown in the graph in FIG. 4E. In line with the above observation, no differences in the number of nestin-positive epSPCs were found between baseline the cultures (gray column) and those treated with miR-124a N/P30 dendriplex (striped column); in contrast, the nestin-positive cells were significantly reduced in the cultures treated with lipofectamine and miR-124a (black column) with respect to the baseline cultures or those treated with miR-124a N/P30 dendriplex. To assess the impact of the dendriplex charged with mimetic miR-124a on epSPC survival at the molecular level and to exclude any excitotoxic effects, the expression levels of the apoptosis-related caspase-6 gene (CASP6) in the same cultures were analyzed. CASP6, which cleaves and activates caspase-3, has been described as a caspase initiator in the apoptotic cascade, leading to neuronal death after an excitotoxic event (Girling et al., 2018, J. Neurosci. Res.).

Total RNA was extracted with TRIzol from 2 to 2.5×10⁵ epSPCs. The RNA quality was verified using a 2100Nano bioanalyzer (Agilent Technologies, Waldbron, Germany). The total RNA was retrotranscribed with TaqMan MicroRNA with miR-124a and U6-specific primers (Thermo Fisher Scientific), the latter as an endogenous control. The cDNA (corresponding to 15 ng of total RNA) was amplified in duplicate by Real Time PCR, using Universal PCR master mix and TaqMan MicroRNA assays (Thermo Fisher Scientific) specific for miR-124a and U6 on Viia7 Real Time PCR System (Applied Biosystem). All the results were normalized with respect to U6 and the relative miRNA expression levels were calculated using the formula 2-ΔCt.

For the gene expression analysis, the total RNA extracted from epSPC, previously screened for miRNA expression, was retro-transcribed using the SuperScript Vilo cDNA synthesis kit (Thermo Fisher Scientific). The cDNA (corresponding to 10 ng of total RNA) was amplified by quantitative real-time PCR, in duplicate, using TaqMan Fast Advanced Master Mix and Taqman gene expression assays (Thermo Fischer Scientific) for caspase-6 (CASP6), cyclin D2 (DIx2) and the 18s housekeeping gene on Viia7 Real-Time PCR (Applied Biosystems). The results are shown in FIG. 4F. In particular, the CASP6 mRNA levels were comparable between cells under baseline conditions (gray column) and those treated with miR-124a N/P30 dendriplex (striped column), but were significantly increased in the epSPC cultures treated with miR-124a and lipofectamine (black column). Confirming the above, these results are indicative of an early activation of apoptotic processes in cells treated with lipofectamine, but not in cells in which miR-124a mimicry was carried out by the dendriplexes according to the present invention. The data confirm the safety of using dendriplexes according to the present invention to mimic miRNA in epSPCs.

1. Fluorinated dendrimeric structure of Formula (I) 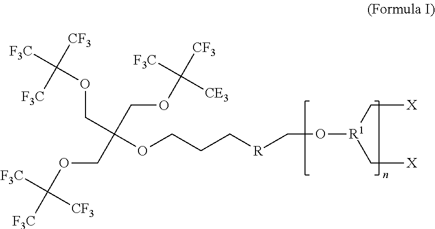
Wherein
n is an integer between 1 and 5;
R is selected from 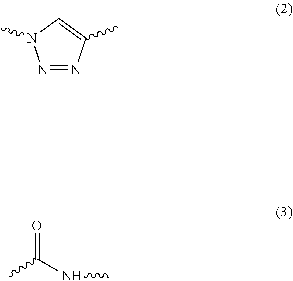
R¹ is selected from 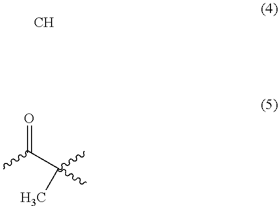
X is selected independently from:
OH; (6)
COOH (7)
NH₂ (8)
NH₃⁺Y⁻ (9)
NR²₂ (10)
NR²₃_{⁺}Y⁻ (11)
Wherein
R² is selected independently from —CH₃, —CH₂CH₃, —CH₂CH₂CH₃, —CH(CH₃)₂, —CH₂OH, —CH₂CH₂OH;
Y⁻ is selected from:

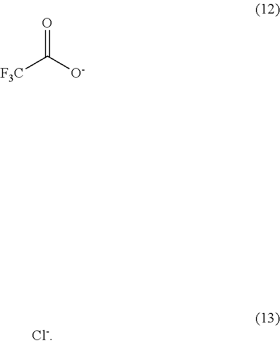

2. The structure according to claim 1, wherein n is 2 or 3.

3. The structure according to claim 1, wherein R is

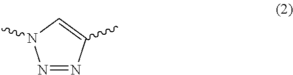

4. The structure according to claim 1, wherein R¹ is

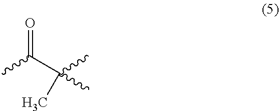

5. The structure according to claim 1, wherein X=NH³⁺Y⁻ and Y is selected from 6.

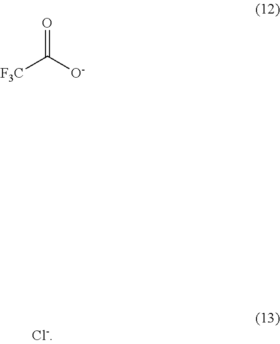

6. The structure according to claim 1, wherein n=3, R is (2), R1 is (5), X=NH₃⁺Y⁻, Y is (12) and it is FDG₂N.

7. A supramolecular complex comprising at least one of the dendrimeric structures according to claim 1 and one or more nucleic acids.

8. The complex according to claim 7, wherein the ratio between the nitrogen atoms of said dendrimeric structure and the phosphorus atoms of said one or more nucleic acids (N/P) is comprised between 5 and 40.

9. The complex according to claim 7, wherein N/P=30.

10. A supramolecular complex according to claim 7, for use in gene therapy.
